(19) United States
(12) Patent Application Publication
(10) Pub. No.: US 2016/0022165 A1
(43) Pub. Date: Jan. 28, 2016

(54) ELECTRODE ASSEMBLIES AND ELECTROENCEPHALOGRAPHS DEVICES

(71) Applicant: ENCEPHALODYNAMICS, INC., Gainesville, FL (US)

(72) Inventors: James C. Sackellares, Gainesville, FL (US); Scott Bearden, Gainesville, FL (US); Ducan Kabinu, Gainesville, FL (US); Michael Poorbaugh, Gainesville, FL (US)

(21) Appl. No.: 14/776,480

(22) PCT Filed: Mar. 14, 2014
(86) PCT No.: PCT/US2014/027804

§ 371 Date: Sep. 14, 2015

Related U.S. Application Data
(60) Provisional application No. 61/783,302, filed on Mar. 14, 2013.

(51) Int. Cl. A61B 5/0478; A61B 5/00

(52) U.S. Cl. CPC A61B 5/0478; A61B 5/6803; A61B 5/742; A61B 2562/164; A61B 2562/168; A61B 2562/225; A61B 2562/0209

(57) ABSTRACT

An electrode assembly can include a reservoir defining a plurality of openings on a first surface of the reservoir, a wicking material extending through one or more of the plurality of openings, and a conductive terminal. An electrode assembly can include a reservoir cap, a reservoir bottom configured to fit to the reservoir cap and define a reservoir therewith, an electrode disposed within the reservoir, and a wicking element in electrical communication with the electrode and configured to absorb a fluid, wherein at least a portion the wicking element is configured to protrude from the reservoir bottom.

### CROSS-REFERENCE TO RELATED APPLICATIONS

This application claims the benefit of and priority to U.S. Provisional Patent Application No. 61/783,302, filed Mar. 14, 2013, the entire contents of which are incorporated by reference herein.

### FIELD

This disclosure related to electrode assemblies, more specifically to electrode assemblies that can be used with electroencephalography.

### BACKGROUND

Electroelectroencephalography is a technology for measuring the voltage and frequency of electrical activity from neurons in the cerebral cortex. Electroencephalogram (EEG) electrodes can record brainwaves using electrodes attached to the scalp, placed on the surface of the brain (subdural electrodes), or within brain tissue (depth electrodes).

A scalp EEG is a non-invasive procedure that provides useful information about brain state and function. This methodology is used in many fields of neuroscience (e.g., psychology, epilepsy, brain machine interface, and sleep research) for recording and analyzing brain state and function. It is used widely as a diagnostic tool in clinical neurology to evaluate and monitor brain function and to identify disturbances in the function of the brain caused by a variety of insults to the brain such as concussion, traumatic injury, stroke, tumor, encephalopathies due to toxins or metabolic disturbances, and seizures. Many disturbances of brain function can be identified by analysis of brief multichannel EEG recordings using electrodes placed in specific locations on the scalp based off reference anatomical landmarks. The most widely accepted system of electrode placement is the International 10/20 System of electrode placement.

### SUMMARY

At least one aspect of this disclosure provides an electrode assembly including a reservoir defining a plurality of openings on a first surface of the reservoir, a wicking material extending through one or more of the plurality of openings, and a conductive terminal.

This aspect of this disclosure can have a variety of embodiments. The wicking material can be porous. The wicking material can include felt and/or foam and/or any other suitable absorptive material.

The electrode assembly can include a conductive fluid absorbed within the wicking material and in contact with the conductive terminal. In some embodiments, the conductive fluid can be saline.

The conductive terminal can be spring-loaded and apply pressure to the conductive fluid. The reservoir can be fabricated from a deformable material such that reservoir deforms when pressed against skin and creates elevated fluid pressure within the reservoir. In some embodiments, the first surface can be substantially flat. Each of the plurality of openings can be each formed on an end of one of a plurality of probes. The plurality of probes can have a height of about 8 mm. The plurality of probes can have a height selected from the group including between about 1 mm and about 2 mm, between about 2 mm and about 3 mm, between about 3 mm and about 4 mm, between about 4 mm and about 5 mm, between about 5 mm and about 6 mm, between about 6 mm and about 7 mm, between about 7 mm and about 8 mm, and between about 8 mm and about 9 mm, or any other suitable height. The plurality of probes can have an external diameter of about 3 mm or any other suitable diameter. In some embodiments, the plurality of probes can have an external diameter selected between about 2 mm and about 4 mm or any other suitable diameter. The plurality of probes can have an internal diameter of about 2 mm or any other suitable diameter.

In some embodiments, the reservoir can define between 5 and 20 openings. In some embodiments, the reservoir can define between 5 and 10 openings.

In some embodiments, the wicking material can extend about 3 mm beyond the plurality of openings. The conductive terminal can be located on an opposite side of the plurality of openings. The conductive terminal can be a silver/silver chloride electrode.

Another aspect of this disclosure provides a device to record brain waves including a headpiece (e.g., an elastic cap, rigid helmet, hat, or other suitable headpiece) adapted and configured for placement on a subject's head, a plurality of electrode assemblies as described herein arranged on an interior surface of the headpiece, and one or more connector cables adapted and configured for coupling with the plurality of electrode assemblies.

This aspect of this disclosure can have a variety of embodiments. The plurality of electrode assemblies can include a plurality of electrode assemblies as described herein located in positions corresponding to regions where the subject does not have hair, and a plurality of electrodes assemblies as described herein located in positions corresponding to regions where the subject does have hair.

In some embodiments, the plurality electrode of assemblies can be located within the headpiece at locations corresponding to the 10-20 System or in any other suitable arrangement. Each of the electrode assemblies can include a mushroom-shaped extension of the conductive terminal that is adapted and configured to snap into one of a plurality of ring-shaped receptacles located on the cap at the locations corresponding to the 10-20 System.

The encephalographic device can include a recording device adapted and configured for coupling with the one or more connector cables and recording electrical signals received from the plurality of electrode symbols via the one or more connector cables.

In some embodiments, the one or more connector cables can be overmolded into the headpiece. The one or more connector cables can include metal wire conductors and conductive cloth.

Another aspect of this disclosure provides an electroencephalographic method including, placing the electroencephalographic recording device as described herein on a subject's head; and recording electrical signals received from the plurality of electrode symbols via the one or more connector cables.

This aspect of this disclosure can have a variety of embodiments. The method can include applying a conductive fluid to the wicking material. The method can include removing a seal from the electrode assemblies. The method can include presenting the electrical signals to a medical professional and/or receiving the electrical signals at a recording and/or monitoring instrument configured to generate a visual tracing of the electrical signals from the various electrodes for interpretation by a user.

Another aspect of this disclosure provides an electroencephalography kit including the encephalographic device as disclosed herein and instructions for use.

In another aspect of this disclosure, an electrode assembly can include a reservoir cap, a reservoir bottom configured to fit to the reservoir cap and define a reservoir therewith, an electrode disposed within the reservoir, and a wicking element in electrical communication with the electrode and configured to absorb a fluid, wherein at least a portion the wicking element is configured to protrude from the reservoir bottom. The wicking element can include a base portion and an array of legs extending therefrom. The wicking element can include a rigid or semi-rigid frame and is coated with a wicking material.

### BRIEF DESCRIPTION OF THE DRAWINGS

Aspects of the present disclosure can be better understood with reference to the following drawings. Components of the drawing are not necessarily to scale, emphasis instead being placed upon clearly illustrating the principles of the present disclosure.

Moreover, like reference numerals designate corresponding parts throughout the several views.

FIG. 1 depicts an electrode assembly designed to be placed on the scalp in areas normally covered with hair according to an embodiment of this disclosure.

FIG. 2 depicts a side cross-sectional view of an electrode assembly according to an embodiment of this disclosure.

FIG. 3A provides a top view of the electrode assembly, illustrating the reservoir 1, the reservoir top 4, and the mushroom-shaped extension 5, which snaps into the electrode cap assembly forming an electrical contact with a trace embedded in the cap assembly according to an embodiment of this disclosure.

FIG. 3B illustrates the undersurface of the electrode assembly, demonstrating the undersurface of the reservoir 1, the hollow contact probes 2, and the tips of the wicking assembly 6 that extend beyond hollow contact probes 2 by approximately 3 mm according to an embodiment of this disclosure.

FIG. 4 illustrates an alternate embodiment of the disclosure wherein the electrode top 4 is modified so as to include a spring 7 made of plastic, metal or other suitable material according to an embodiment of this disclosure.

FIG. 5 illustrates an elastic cap assembly made from an elastic material such as spandex, which is sized to fit over a human head. FIG. 5A provides a lateral view and FIG. 5B provides a frontal view of the cap assembly according to an embodiment of this disclosure.

FIG. 6 shows a cutout of the exterior surface of the elastic cap 12 illustrating the attachment of an example electrode assembly 11 of FIG. 5A according to an embodiment of this disclosure.

FIG. 7 is a side view of the electrode assembly and electrode top, illustrating how one of a plurality of electrode assemblies is attached to the undersurface of the elastic cap 12 in a typical embodiment of this disclosure according to an embodiment of this disclosure.

FIG. 8 depicts an alternate embodiment of this disclosure useful for electrodes that overlie scalp that is typically not covered with hair (e.g., Fp1, Fp2, F3, F4, F7, and F8 in the International 10/20 System of Electrode Placement) according to an embodiment of this disclosure.

FIG. 9 depicts an encephalographic method 900 according to an embodiment of this disclosure.

FIG. 10 depicts an electroencephalography kit 1000 including an electroencephalography device 1002 and instructions for use 1004 according to an embodiment of this disclosure.

FIG. 11 depicts the location of electrodes in the International 10/20 System of electrode placement.

FIG. 12 is a perspective view of another embodiment of an electrode assembly in accordance with this disclosure.

FIG. 13 is an exploded view the electrode assembly of FIG. 12.

FIG. 14 is a cross-sectional view the electrode assembly of FIG. 12.

### DETAILED DESCRIPTION

As used in the specification and claims, the singular form “a,” “an,” and “the” include plural references unless the context clearly dictates otherwise. Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “comprises,” “comprising,” “containing,” “having,” and the like can have the meaning ascribed to them under U.S. patent law and can mean “includes,” “including,” and the like.

A “health care professional” shall be understood to mean any person providing medical care to a patient. Such persons include, but are not limited to, medical doctors, physician's assistants, nurse practitioners (e.g. an Advanced Registered Nurse Practitioner (ARNP)), nurses, residents, interns, medical students, or the like. Although various licensure requirements may apply to one or more of the occupations listed above in various jurisdictions, the term health care provider is unencumbered for the purposes of this patent application.

Unless specifically stated or obvious from context, the term “or,” as used herein, is understood to be inclusive.

A “subject” shall be understood to include any mammal including, but not limited to, humans.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

The diagnosis of brain disorders is aided by many technologies such as magnetic resonance imaging (MRI), computerized axial tomography (CT scan), functional MRI (fMRI), positron emission tomography (PET) and magnetoencephalography (MEG). MRI and CT scan provide images of brain structure, PET images brain metabolism or blood flow, and MEG images neuronal function. However, none of these methods can be done at the bedside, due the size of the instruments or the need for special environments.

The EEG is the test of choice for evaluating patients presenting with altered mental status because it is the only current diagnostic tool for evaluating brain function that provides direct information regarding brain neuronal function and can be performed at the bedside. For this reason, it is widely used as a portable diagnostic tool in urgent care and emergency settings as well as in a laboratory environment. However, the diagnostic use of the EEG is limited by the difficulty in applying the multiple sensing electrodes required in the proper anatomical locations on the scalp in a way that ensures excellent and secure electrical contact. The proper application of EEG electrodes is particularly difficult to achieve in areas of the scalp normally covered by hair. Thus, proper application of EEG electrodes for diagnostic purposes must be performed by highly trained electroneurodiagnostic technicians. Even under ideal conditions, such as in an EEG laboratory, proper application of the electrodes by a skilled technician requires approximately 20 minutes. Due to the limited availability of trained technicians and the cost of ensuring their availability around the clock, the use of EEG in emergency and urgent care environments is limited primarily to large tertiary care hospitals.

Aspects of the this disclosure provide an electrode assembly and an electrode cap assembly designed to enable rapid application of a set of EEG electrodes (typically 21 electrodes) in the proper anatomical location on the scalp in such a way as to achieve reliable low impedance electrical contact with the scalp. Using this device along with standard portable EEG recording devices, an EEG can be obtained anytime and anywhere (e.g., in emergency departments, emergency vehicles, and in other acute care settings) by individuals who are not trained electroneurodiagnostic technicians.

At least one embodiment of this disclosure provides a plurality of novel electrode assemblies (e.g., 21 in some embodiments) that are attached to a headpiece (e.g., an elastic cap, rigid helmet, hat, or other suitable headpiece) at specified locations (through a snap mechanism in a typical embodiment), conductive traces (typically metal wire conductors, conductive cloth or other conductive materials), and electrical connections used to connect the electrode set with an EEG recording device. The electrode includes a reservoir configured to be filled with a conductive electrolyte fluid (e.g., saline solution, an electrolyte gel, or any other suitable conductive substance), a wicking material designed to control the flow of conducting fluid, and a reservoir top constructed from conductive material (e.g. silver/silver chloride disk or conductive rubber). The reservoir has multiple contact probe extensions designed to protrude through the hair to the scalp. The wicking material is a spongy material (e.g., foam in a typical embodiment) that sits within the reservoir and has probe extensions that fit within the probes attached to the reservoir. The wick ensures that the conductive fluid is in continuous contact with the scalp, thus maintaining electrical contact between the scalp and the electrode assembly.

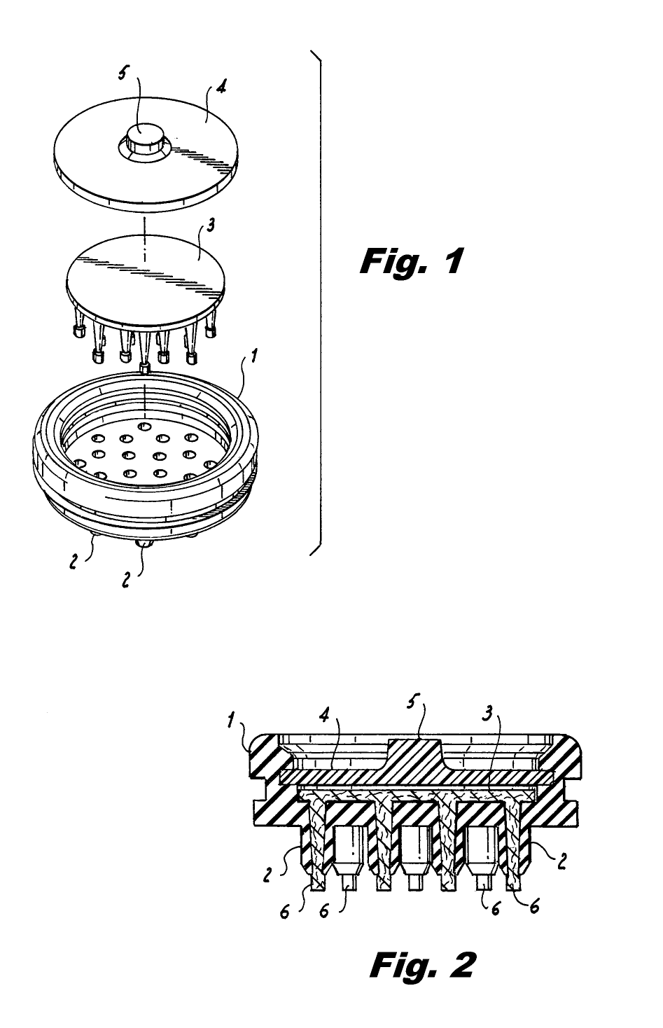

FIG. 1 depicts an embodiment of an electrode assembly designed to be placed on the scalp in areas normally covered with hair. It is also contemplated that the embodiment of FIG. 1 can be used with areas not having hair. FIG. 1 is an exploded view depicting the key components of the electrode assembly that includes a hollow reservoir 1 made of a compressible material. In a typical embodiment, this reservoir 1 is fabricated from a conductive elastomeric material.

The reservoir 1 is configured to contain conducting fluid, such as a saline solution, a conductive gel, or any other suitable fluid. The undersurface of the reservoir 1 includes a flat surface with multiple holes and hollow contact probes 2 that penetrate the hair, facilitating electrical contact with the scalp. These hollow contact probes 2 can be aligned with the holes in the undersurface of the reservoir, providing a continuous path for flow of an electrolyte fluid between the reservoir 1 and the contact probes 2. In some embodiments embodiment, each of the contact probes 2 is approximately 8 mm in length with an outside diameter of 3 mm and an inside diameter of 2 mm.

In some embodiments, the elastomeric material of the reservoir 1 is made of a rubber material which may be conductive or insulating. This material can be compressible such that compression by the elastic cap 12 against the reservoir 1 creates sufficient pressure within the reservoir 1 to cause the fluid to pass from the reservoir 1 through the probes 2 to the scalp. This maintains sufficient pressure to ensure that the tips of the wicking material 6 remain wet, thus maintaining electrical contact with the scalp. Also, the compressible material of the reservoir 1 provides more comfort to the subject or patient under a wider range of conditions. It is contemplated that reservoir 1 can be rigid and that the wicking material 6 can remain wet via capillary action.

The second component of the electrode assembly is a wicking module 3 made of a porous material, such as foam or felt. The wicking material serves to hold the conducting fluid within the reservoir and hollow contacts, restricting the free-flow of the fluid sufficiently to wet the surface of the scalp beneath the probe 2 while preventing emptying of the reservoir 1 through leakage. The wicking material is pre-formed to fit within the reservoir 1 and has probes 6 that extend into the hollow contact probes 2, extending approximately 3 mm past the end of the hollow probes 2.

The third component of the electrode assembly is a reservoir top 4. This top 4 is typically constructed from a conductive metal, such as metal (e.g., silver/silver chloride) or an elastomeric conductive material, such as that used for the reservoir. The top 4 seals the conducting fluid within the reservoir 1 and is in direct contact with the wicking material 3, facilitating electrical contact through the electrolyte solution. In the center of the upper surface of the top 4 is a mushroom-shaped extension 5, which snaps into an electrode cap assembly, or other device such as flexible strips, to attach each electrode to the cap assembly. The center of the mushroom top 5 is a self-sealing needle port, typically constructed of rubber, which allows the user to refill the electrolyte solution, if required.

Referring now to FIG. 2, a side cross-sectional view of the hair electrode assembly illustrates how the wick probes 6 (which are part of the wicking module 3) extend from the reservoir 1 into and beyond the hollow contact probes 2 of the reservoir 1. FIG. 2 also illustrates that the reservoir top 5 can be recessed within reservoir 1.

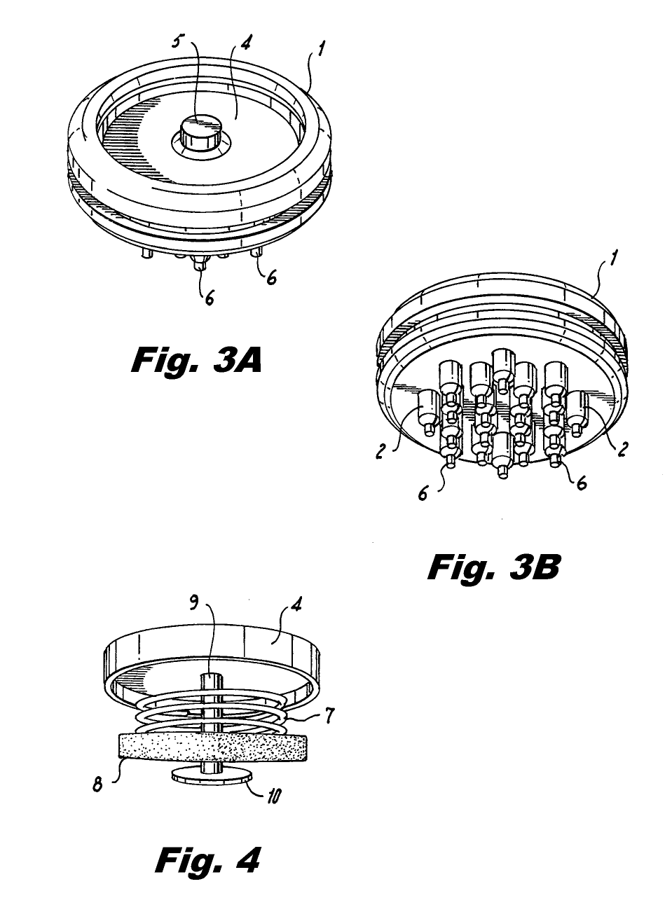

FIG. 3A provides a top view of the electrode assembly, illustrating the reservoir 1, the reservoir top 4, and the mushroom-shaped extension 5, which snaps into the electrode cap assembly forming an electrical contact with a trace embedded in the cap assembly.

FIG. 3B illustrates the undersurface of the electrode assembly, demonstrating the undersurface of the reservoir 1, the hollow contact probes 2, and the tips of the wicking assembly 6 that extend beyond hollow contact probes 2 by approximately 3 mm.

FIG. 4 illustrates an alternate embodiment of the disclosure wherein the electrode top 4 is modified so as to include a spring 7 made of plastic, metal or other suitable material. Below the spring 7 is an O-ring diaphragm 8, fabricated from rubber or other suitable material. These components are held in place by a shaft 9 made of metal or other electrical conducting material. A silver/silver chloride electrode 10 is attached to the inferior portion of the shaft 9. The spring 7 exerts pressure on the O-ring/diaphragm, which compresses the electrolyte fluid (e.g. normal saline) and the wicking material in the reservoir 1. Thus, as the fluid level declines in the reservoir 1 over time, the fluid passes from the reservoir 1 through the probes 2 to the scalp, the spring 7 pushes against the diaphragm 8 and the top 4, acting as a piston to maintain a steady pressure within the reservoir 1 to ensure that the tips of the wicking material 6 remain wet, thus maintaining electrical contact with the scalp.

In a third alternative embodiment, a hybrid reservoir configuration can be constructed from a non-compressible (e.g., hard rubber or plastic) material containing a spring assembly 7 and a O-ring diaphragm 8 as shown in FIG. 4 and discussed above. This material can constitute the upper ⅔ of the chamber. The lower ⅓ of the reservoir is composed of compressible elastomeric material as discussed above. This embodiment combines the adjustable pressure of the spring piston with the comfort and flexibility of the rubber chamber.

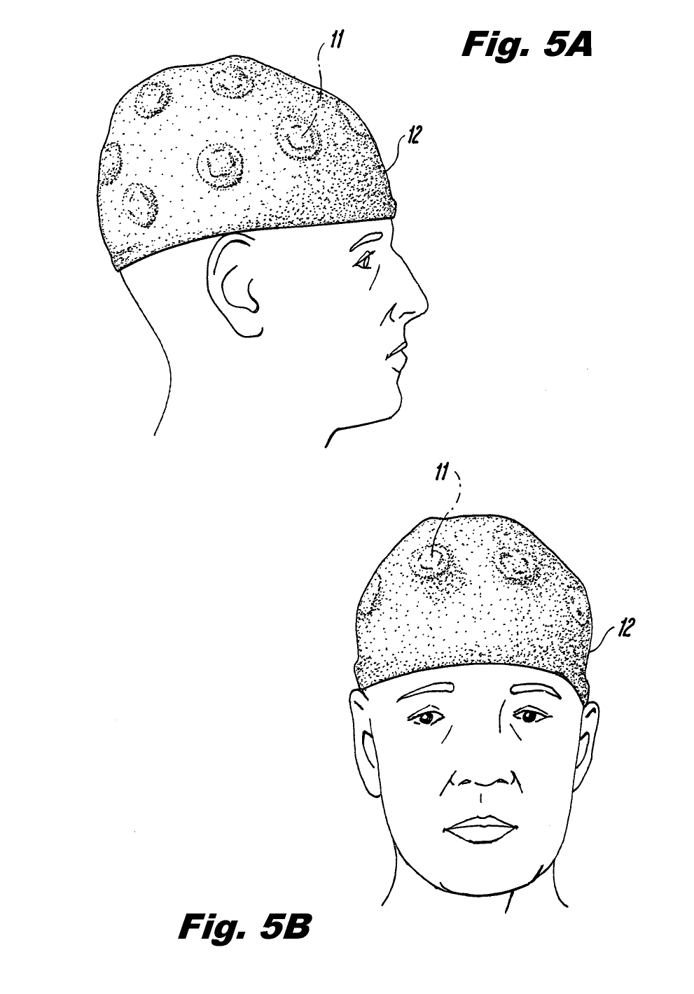

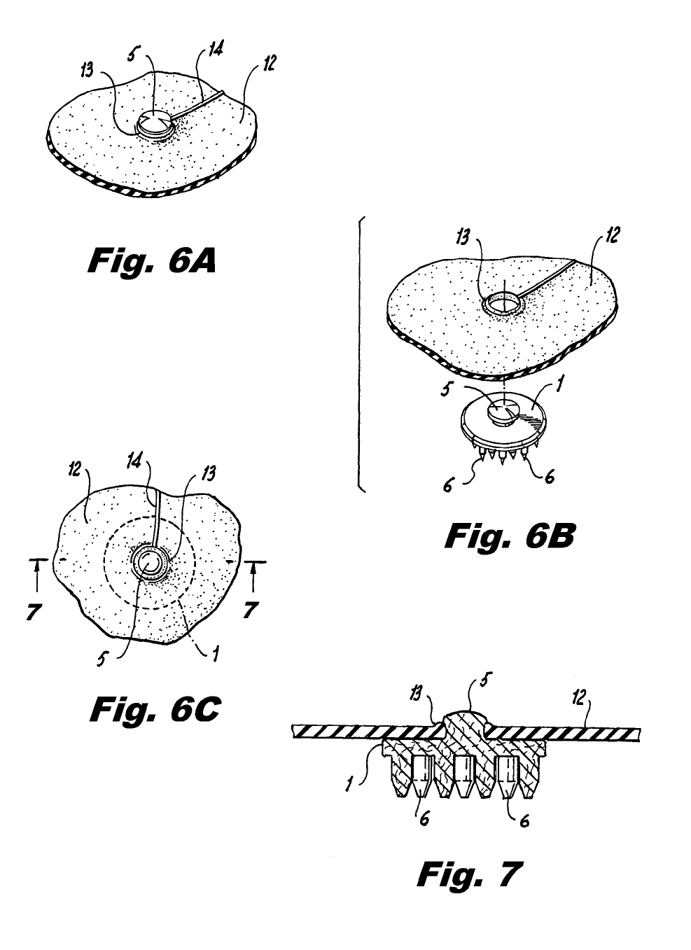

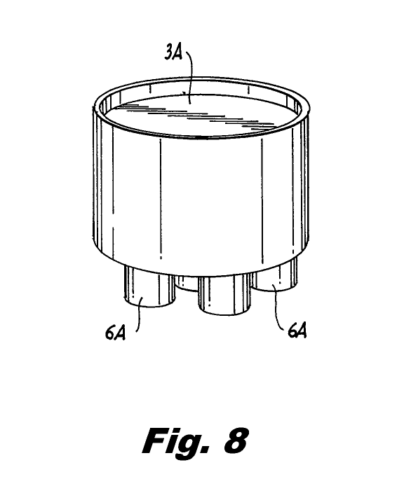

FIG. 5 illustrates an elastic cap assembly made from an elastic material such as spandex, which is sized to fit over a human head. FIG. 5A provides a lateral view and FIG. 5B provides a frontal view of the cap assembly. The size of the cap varies, so as to be used for adults, children of various ages, and infants. A plurality of electrode assemblies 11 are attached to the undersurface of the cap, by snapping the mushroom shaped extension of each electrode assembly 5, as shown in FIG. 6.

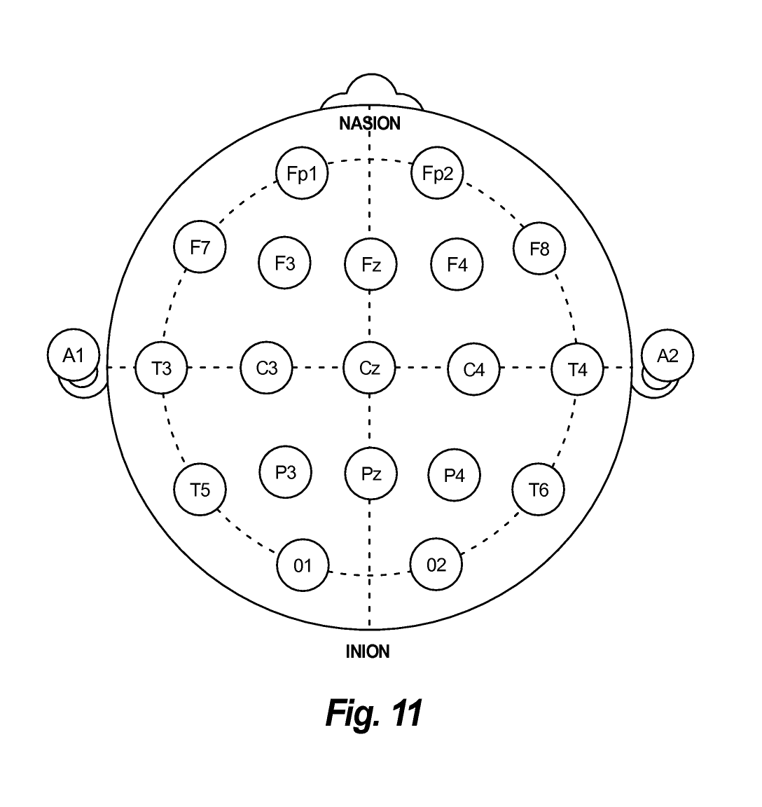

Electrode assemblies can attach to the electrode cap through snap mechanisms depicted in FIG. 6, which can be attached to holes placed in the cap at specified distances. Any other suitable attachment is contemplated. The distances between holes in the cap are determined so that the electrode assemblies come into contact with the scalp at any suitable desired locations. In some embodiments, the inter-hole distances are set so that the electrode assemblies contact the scalp in anatomical locations approximating those of the International 10/20 System of electrode placement, which is depicted in FIG. 11. Typically, 21 electrodes are attached to the cap, so as to have 5 left parasagittal electrodes, 5 right parasagittal electrodes, 3 midline electrodes, 3 left temporal electrodes and 3 right temporal electrodes, as well as a ground and common reference electrode. The cap can be modified to include additional electrodes (typically for research purposes) or to include fewer electrodes, as required for the specific clinical or research application.

FIG. 6 shows a cutout of the exterior surface of the electrode cap 12 illustrating the attachment of an example electrode assembly 11. The mushroom-shaped extension of the electrode top 5 snaps into a ring-shaped receptacle 13, which is attached to holes in the cap. In a typical embodiment, the receptacle may be sewn into place on the cap. The receptacle ring is fabricated from a conductive material such as metal or a conductive elastomeric material and is attached to a conductive trace 14. The trace 14 may be a metal wire, conductive cloth or other suitable electrical conducting material. In one embodiment, the conductive trace is overmolded into the elastic cap. The conductive trace 14 from each electrode assembly continues to a multi-contact electrical connector which, in turn, is plugged into an EEG recording device. The configuration of the multi-contract connector can be modified to be compatible with any given recording device.

FIG. 7 is a side view of the electrode assembly and electrode top, illustrating how at least one of a plurality of electrode assemblies can be attached to the undersurface of the elastic cap 12 in at least one embodiment of this disclosure. The mushroom-shaped extension of the electrode top 5 protrudes through a hole in the elastic cap material and is fixed by snapping into the receptacle 13. As shown, the reservoir 1 can be located on the undersurface of the elastic cap.

The foregoing description illustrates some embodiments of an electrode configured to be used in areas of the scalp that are usually covered with hair, although it is contemplated that such embodiments can be used with areas of tissue not covered with hair. FIG. 8 depicts an alternate embodiment of this disclosure useful for electrodes that overlie scalp that is typically not covered with hair (e.g., Fp1, Fp2, F3, F4, F7, and F8 in the International 10/20 System of Electrode Placement), although it is contemplated that such embodiments can also be used in locations with hair. Such embodiments can include the three basic components discussed in the context of FIG. 1 (i.e., a hollow reservoir 1, wicking module 3, and reservoir top 4). However, the modified electrode would not include hollow contact probes 2 extending from the undersurface of the reservoir 1. This modification may be preferable in areas without hair because contact probe points are not required to establish contact through the hair. In such embodiments, the wicking material probes 6A would extend approximately 3 mm beyond the undersurface of the reservoir. Alternatively, the wicking material 3A may not have probes 6A, but there would be perforations in the underside of the reservoir 1 to allow the electrolyte to maintain contact with the scalp.

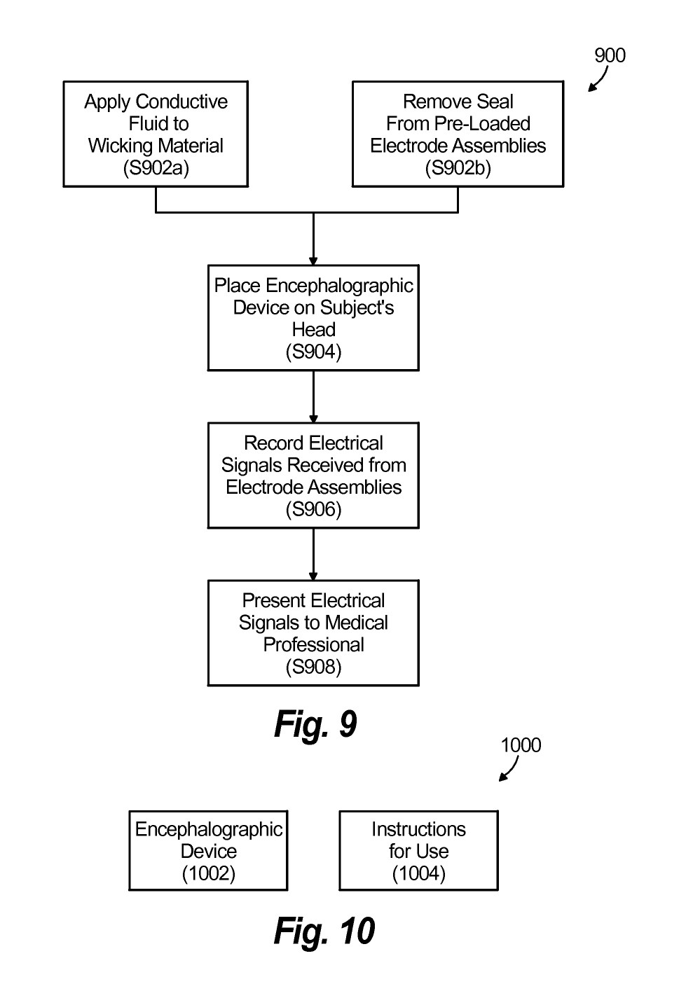

Referring now to FIG. 9, another aspect of this disclosure provides an electroencephalographic method 900. In step S902a, a conductive fluid can optionally be applied to the electrode assemblies as discussed herein. In other embodiments, the electrode assemblies can be previously-loaded with conductive fluid. In some embodiments, the electrode assemblies are provided in sealed pouches that ensure sterility and minimize fluid loss from the electrode assemblies. Thus, in step S902b, a seal can be removed to allow fluid flow from a previously-loaded electrode assembly. In step S904, an encephalographic device is placed on the subject's head. The encephalographic device can be an encephalographic device as described herein. The encephalographic device can be marked in order to facilitate proper orientation of the encephalographic device with respect to the subject's head.

In step S906, electrical signals are received from the electrode assemblies and can be recorded (e.g., in computer readable media or on paper). After receiving the electrical signals, a recording and/or monitoring instrument can be configured to generate a visual tracing or other suitable display of the electrical signals from the various electrodes for interpretation by a user. In step S908, the electrical signals and/or the visual tracing (or other suitable display) are presented to a medical professional. The electrical signals can be presented to the medical professional in a variety of formats including in a paper report, in a computer readable file, on an electronic display, and the like.

Referring now to FIG. 10, another aspect of this disclosure provides an electroencephalography kit 1000 including an electroencephalography device 1002 and instructions for use 1004. The electroencephalography device 1002 can be an electroencephalography device as described herein. The instructions for use 1004 can be in written or electronic form and can include, for example, instructions on how to position the encephalographic device 1002 on the subject's head, how to couple the electroencephalography device 1002 to appropriate hardware for storing, displaying, and/or interpreting the electrical signals received from the electrode assemblies, general or specific instructions (e.g., word descriptions, illustrations, symbols) regarding methods of using the device, and collecting and measuring data.

Aspects of this disclosure can be particularly useful for detection and diagnosing concussions. For example, the electroencephalography kits can be stocked in ambulances for application while a patient is being transported to the hospital after a traffic accident. Likewise, the electroencephalography kits can be used by team doctors, trainers, and other medical professionals to quickly assess whether an athlete sustained a concussion in sports such as football, soccer, and hockey. In particular, embodiments of this disclosure can be worn under football or hockey helmets and automatically monitored via wireless communication to automatically detect concerning electrical signals.

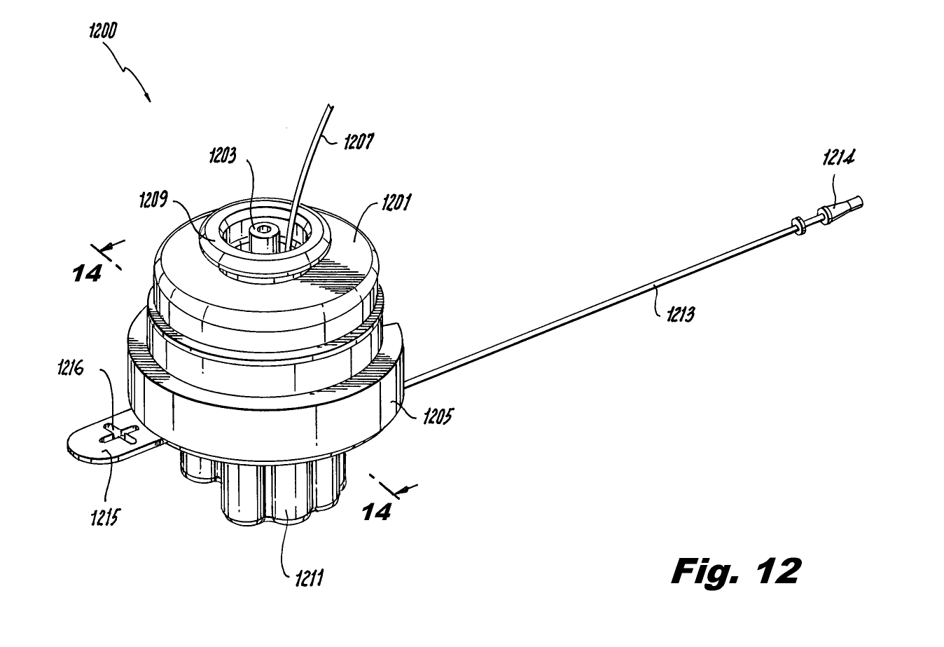

Referring to FIGS. 12-14, another embodiment of an electrode assembly 1200 is shown. Electrode assembly 1200 includes a reservoir cap 1201 that defines an interior cavity. A reservoir bottom 1327 is configured to be connected, moveably attached, and/or releasably attached to the reservoir cap 1201 in any suitable manner (e.g., bonding, bolting, molding, adhesives). The reservoir cap 1201 and the reservoir bottom 1327 define a reservoir 1350 that is configured to include at least one absorptive pad 1317, at least one electrode 1319, and at least a portion of a wicking element 1321.

The reservoir cap 1201 can be formed a substantially rigid material (e.g., polypropelene) or any other suitable material, including any suitable semi-rigid materials. The reservoir cap 1201 can define a fill port 1203 configured to be in fluid communication with the reservoir 1350. In some embodiments, the reservoir cap 1201 can also define and/or include an attachment portion (e.g., button attachment 1209) that is configured to allow the device to attach to a cap for a human head. The reservoir cap 1201 can also include a hole for one or more electrode wires 1207 and/or allow one or more electrode wires 1207 to exit through the fill port 1203.

The reservoir bottom 1327 can form and/or include one or more hollow contact probes 1329 extending therefrom which are in communication with holes 1331 define in the reservoir bottom. In some embodiments, the hollow contact probes 1329 are separate from the reservoir bottom 1327 and can be fixedly or removably attached and/or be interchangeable with different sets of hollow contact probes 1329. The hollow contact probes 1329 can be any suitable length, and may include differing lengths between one or more of the probes. In some embodiments, such as one intended for use with a portion of skin having hair, the hollow contact probes 1329 can be about 9 mm long. In some embodiments, such as one intended for use with a portion of skin not having hair, the hollow contact probes 1329 can be about 3 mm long.

The reservoir bottom 1327 can be made of any suitable rigid, semi-rigid, or flexible material (e.g., elastomeric plastic). If the reservoir bottom 1327 is configured to be semi rigid or flexible, the reservoir bottom 1327 can provide more comfort and flexibility for contact with skin at a test site (e.g., a scalp). It is also contemplated that the hollow contact probes 1329 can be made of a softer and/or more flexible material than the reservoir bottom 1327.

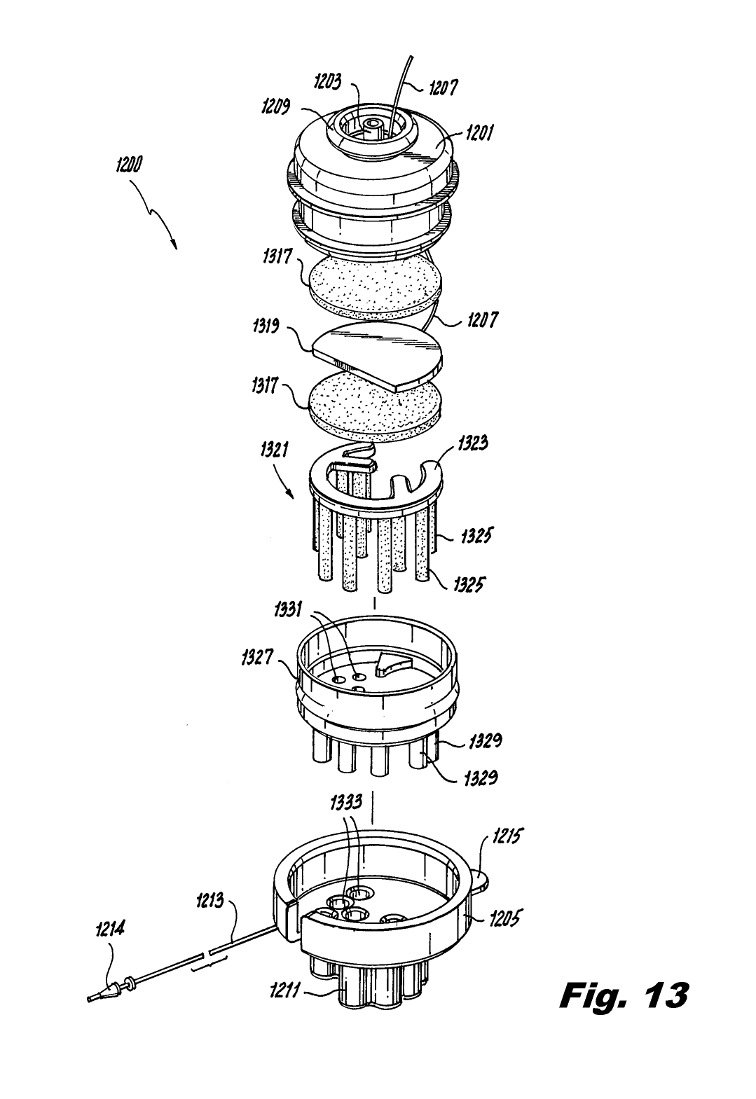

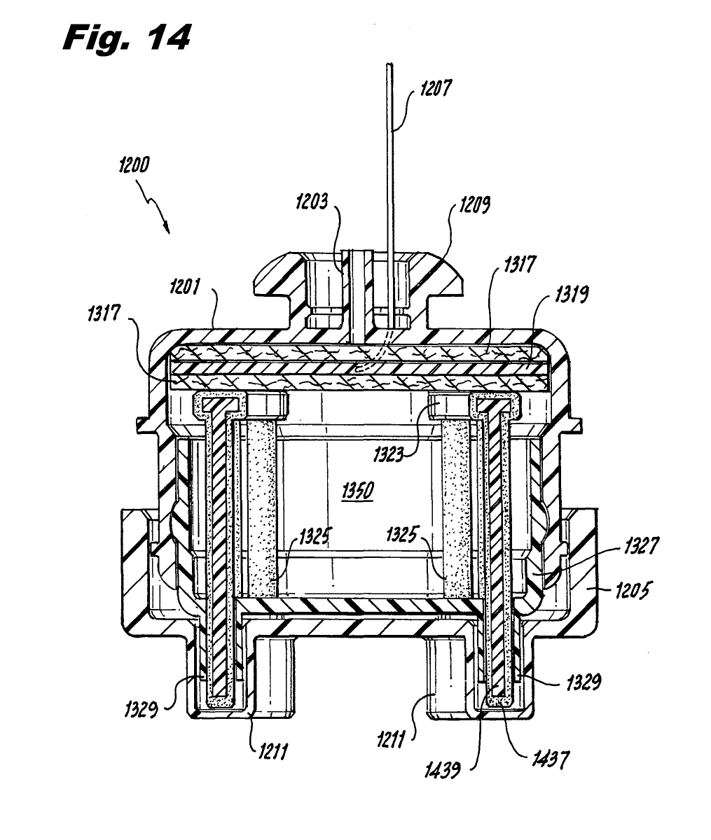

An electrode 1319 can be disposed within the reservoir 1350 and be connected to the one or more electrode wires 1207. As shown in FIGS. 13 and 14, the electrode can include any suitable shape that allows fluid to flow around a portion of the electrode 1319 such that the fluid can reach the bottom of the reservoir 1350. Also as shown in the embodiments of FIGS. 12-14, the electrode 1319 can be in contact (e.g., sandwiched between as shown) with one or more absorptive pads 1317. The electrode 1319 can be made of any suitable material (e.g., Silver-Silver-Chloride).

The absorptive pads 1317 can be any suitable absorptive material (e.g., a spongy material) that is configured to absorb a conductive fluid. The absorptive pads 1317 can be any suitable shape, thickness, and/or size and are not necessarily disk shaped as shown. The one or more absorptive pads 1317 can be configured to fill out any remaining interior space of the reservoir 1350.

The wicking element 1321 can include any suitable absorptive material similar to the absorptive pads. In some embodiments, the wicking element 1321 can include a rigid or semi-rigid frame 1439 having an absorptive layer 1437 disposed thereon. The absorptive layer 1437 can be deposited on the frame 1439 in any suitable manner (e.g., flocking).

The wicking element 1321 can include a base portion 1323 and one or more legs 1325 extending therefrom. The legs 1325 can be of any suitable length, shape, and/or width/diameter. In some embodiments, the frame 1439 of at least one of the legs 1325 can include a width of about 1 mm. The legs 1325 can be arranged in any suitable manner and/or form any suitable pattern or array. Disposing the legs 1325 in an array allows for simple manufacture and installation of the wicking element 1321 within the contact probes 1329.

The absorptive layer 1437 can be any suitable thickness (e.g., about 0.4) mm thick on at least a portion of the wicking element 1321. The legs 1325 are configured to enter into holes 1331 of the reservoir bottom 1327 and be contained in and/or protrude at least partially out of hollow contact probes 1329. In this respect, the legs 1325 are configured to contact the skin tissue of a patient and transmit electrical signals to the electrode 1319.

When assembled, the electrode assembly 1200 can be filled with a fluid through fill port 1203. The fluid can enter into the reservoir 1350 and be absorbed by the absorptive pads 1317. Through gravity and/or capillary action, the fluid within the pads and/or excess unabsorbed fluid can travel to the wicking element 1321 and be distributed into the absorptive layer 1437 and down throughout the legs 1325. In this respect, the legs 1325 stay wetted and allow for a continuous electrical connection to the electrode 1319.

In some embodiments, the electrode assembly 1200 can include a probe cover 1205 configured to overlay and/or seal the hollow contact probes 1329 to store the electrode assembly with fluid therein without allowing the wicking element 1321 to dry out. The probe cover 1205 can include sealed channels 1211 extending therefrom and holes 1333 for inserting the hollow contact probes 1329 into the sealed channels 1211. Each prober cover 1205 may include a daisy chain system including a shaft member 1213 having a male portion 1214 and a tab member 1215 having a female portion 1216. The male portion 1214 is configured to be inserted and locked to the female portion 1216 such that, when daisy chained, removing a first probe cap 1205 requires the removal of any daisy chained probe caps 1205.

As disclosed herein, embodiments of the electrode assembly act as a fluid moving system that enables one to deliver a conductive electrolytic solution to the scalp through hair, so as to saturate the high impedance outer layer of the epidermis, thereby lowering impedances to levels that are acceptable for clinical diagnostic purposes (e.g., below 10 K Ohms) There should be an adequate flow of fluid to saturate the epidermal layer, but not an excessive flow of the fluid since too much flow can create recording problems and/or prematurely dry out the wicking material and the reservoir.

Although embodiments of this disclosure have been described using specific terms, such description is for illustrative purposes only, and it is to be understood that changes and variations may be made without departing from the spirit or scope of the following claims.

1. An electrode assembly comprising:
a reservoir defining a plurality of openings on a first surface of the reservoir; a wicking material extending through one or more of the plurality of openings; and a conductive terminal.

2. The electrode assembly of claim 1, wherein the wicking material is porous.

3. The electrode assembly of claim 1, wherein the wicking material includes at least one of felt or foam.

4. The electrode assembly of claim 1, further comprising:
a conductive fluid absorbed within the wicking material and in contact with the conductive terminal.

5. The electrode assembly of claim 4, wherein the conductive fluid is saline.

6. The electrode assembly of claim 4, wherein the conductive terminal is spring-loaded and applies pressure to the conductive fluid.

7. The electrode assembly of claim 4, wherein the reservoir is fabricated from a deformable material such that reservoir deforms when pressed against skin and creates elevated fluid pressure within the reservoir.

8-19. (canceled)

20. An electroencephalographic device comprising:
a headpiece adapted and configured for placement on a subject's head;
a plurality of electrode assemblies according to claim 1 arranged on an interior surface of the headpiece; and
one or more connector cables adapted and configured for coupling with the plurality of electrode assemblies.

21. The electroencephalographic device of claim 20, wherein the plurality of electrode assemblies include:
a plurality of electrode assemblies according to claim 8 located in positions corresponding to regions where the subject does not have hair; and
a plurality of electrodes assemblies according to claim 9 located in positions corresponding to regions where the subject does have hair.

22. The electroencephalographic device of claim 20, wherein the plurality of electrode assemblies are located within the headpiece at locations corresponding to the 10-20 System.

23. The electroencephalographic device of claim 22, wherein the each of the electrode assemblies includes a mushroom-shaped extension of the conductive terminal that is adapted and configured to snap into one of a plurality of ring-shaped receptacles located on the cap at the locations corresponding to the 10-20 System.

24. The electroencephalographic device of claim 20, further comprising:
a recording device adapted and configured for coupling with the one or more connector cables and recording electrical signals received from the plurality of electrode symbols via the one or more connector cables.

25. (canceled)

26. The electroencephalographic device of claim 20, wherein the one or more connector cables are selected from the group consisting of: metal wire conductors and conductive cloth.

27. An electroencephalographic method comprising:
placing the electroencephalographic recording device of claim 20 on a subjects head; and
recording electrical signals received from the plurality of electrode symbols via the one or more connector cables.

28. The method of claim 27, further comprising:
applying a conductive fluid to the wicking material.

29. The method of claim 27, further comprising:
removing a seal from the electrode assemblies.

30. The method of claim 27, further comprising:
receiving the electrical signals at a recording and/or monitoring instrument configured to generate a visual tracing of the electrical signals from the various electrodes for interpretation by a user.

31. An electrode assembly, comprising:
a reservoir cap;
a reservoir bottom configured to fit to the reservoir cap and define a reservoir therewith;
an electrode disposed within the reservoir; and
a wicking element in electrical communication with the electrode and configured to absorb a fluid, wherein at least a portion the wicking element is configured to protrude from the reservoir bottom.

32. The electrode assembly of claim 31, wherein the wicking element includes a base portion and an array of legs extending therefrom.

33. The electrode assembly of claim 31, wherein the wicking element includes a rigid or semi-rigid frame and is coated with a wicking material.
